(19) United States
(12) Patent Application Publication
(10) Pub. No.: US 2025/0299770 A1
(43) Pub. Date: Sep. 25, 2025

(54) METHOD AND SYSTEM FOR SEARCHING KEY ATOM SET IN FUNCTIONAL DYNAMICS OF BIOMOLECULES

(71) Applicants: THE CHINESE UNIVERSITY OF HONG KONG, SHENZHEN, SHENZHEN (CN); The Chinese University Of Hong Kong, Shenzhen Futian Biomedical Innovation R&D Center, SHENZHEN (CN)

(72) Inventors: LIZHE ZHU, SHENZHEN (CN); KUN XI, SHENZHEN (CN)

(21) Appl. No.: 19/230,128

(22) Filed: Jun. 6, 2025

Related U.S. Application Data
(63) Continuation of application No. PCT/CN2023/108237, filed on Jul. 19, 2023.

(30) Foreign Application Priority Data
Dec. 8, 2022 (CN) 202211568279.4

(51) Int. Cl. G16B 15/00; G06F 30/27; G16B 40/20

(52) U.S. Cl. CPC G16B 15/00; G06F 30/27; G16B 40/20

(57) ABSTRACT

The present disclosure provides a method and system for searching a key atom set in functional dynamics of biomolecules. The method includes: performing optimization to obtain a transition path based on an initial state and a target state, and recording M transition structures; based on a difference between the initial state and the target state, extracting Cα atoms, and atoms capable of forming a hydrogen bond, salt bridge or π-π stacking interaction, to construct a reference atom set; screening out atoms capable of forming a hydrogen bond, salt bridge or π-π stacking interaction with atoms of the reference atom set from the M transition structures to construct a supplementary atom set; and screening out unstable atoms from the supplementary atom set by evaluating the stability of interaction between the reference atom set and the supplementary atom set, and performing integration to obtain a key atom set.

### TECHNICAL FIELD

The present disclosure relates to the field of studying computational simulation of biomolecular systems, and in particular, to a method and a system for searching a key atom set in functional dynamics of biomolecules.

### BACKGROUND

The achievement of biomolecular functions is often accompanied by complex structural transition processes, which is referred to as functional dynamics of biomolecules. The specific structural transition process (i.e., a transition mode, also referred to as a transition path) mainly occurs through a minimum free energy pathway (MFEP). Therefore, various algorithms have been developed to efficiently search the MFEP, thereby accurately grasping the details of changes in biomolecular structures. A TAPS algorithm has been developed, which is less dependent on input information and can automatically obtain a high-dimensional description of the MFEP (including almost all atoms of biological macromolecules). However, after the MFEP is obtained, it is necessary to obtain a free energy surface distributed along the MFEP to further determine the important transition state and metastable state information in the process, so as to fully explain the transition mechanism of functional dynamics of biomolecules.

Therefore, how to quickly and accurately obtain the free energy surface of the transition path is extremely critical. Given an existing high-dimensional MFEP, the transition process, which originally needs to be described in a high-dimensional space, may be reduced to two dimensions by defining a path collective variable (PCV). Specifically, the transition may be referenced along a transition path direction (PCV-s) and perpendicular to a transition path (PCV-z). The definition of PCV relies on a distance d that describes a difference between high-dimensional structures, and it is necessary to determine in advance which atoms are used to calculate d. Therefore, computing the free energy surface of the MFEP requires an atom set as an input parameter, and the quality of this subset of atoms determines the accuracy of the resulting free energy surface and the underlying transition mechanism thereof. However, there is currently no efficient and general atom set screening method to address the above challenges. Therefore, developing an automated searching algorithm for key residues (atom sets) in transition paths based on the important interactions between atoms/residues in biomolecules becomes one of the feasible solutions to address the above challenges.

The current method mainly examines the interactions between all residues of biomolecules and obtains a target atom set through comparative analysis. Considering that the number of biomolecular residues is large (on the order of 101-103), and the transition path of the biomolecular residues often includes a plurality of intermediate structures (on the order of 101-102). Meanwhile, to accurately obtain dominant key residues in the transition path, it is also necessary to fully examine residue differences between any adjacent structures in the transition path (the order of the interaction that needs to be examined increases to 103-108). Therefore, there is currently no efficient and mature computational method that can accurately obtain the key residues in the transition path of functional dynamics of biomolecules.

According to existing study method, it is necessary to reduce the number of residues that need to be examined in advance (to the order of 101) based on studying experience or preliminary analysis, and then determine the key residues by the same residue interaction analysis to obtain the target atom set. This previously summarized experience method for reference has a clear applicable system, and the process is shown in FIG. 1 of the specification. The most important step is to count interaction differences between any adjacent residues in a local region along the transition path, and screen out key interactions and important residues. The screening still needs to be manually extracted based on experience. Based on the screened key residues, this method attempts to explain the transition mechanism of the functional dynamics of biomolecules through changes in interactions of the key residues; if the explanation of the relevant mechanism is incomplete, new residues need to be supplemented and manual screening and interpretation need to be repeated.

It is easy to find that when the transition path of functional dynamics of biomolecules involves the transition of a plurality of structural domains, that is, the number of residues and interactions that need to be examined is too large, the method of screening based on experience consumes a huge time cost. For this reason, this method is also limited to the stable application of the study of dominant key residues in the mechanism functional dynamics of complex biomolecules.

### SUMMARY

In view of this, the present disclosure provides a method and a system for searching a key atom set in functional dynamics of biomolecules. The specific solution is as follows:

A method for searching a key atom set in functional dynamics of biomolecules includes:
performing optimization to obtain a corresponding transition path based on an initial state and a target state of functional dynamics of biomolecules, and recording M transition structures generated in the optimization process and including the initial state and the target state; wherein M is a natural number greater than 10;
by comparing a structural difference between the initial state and the target state, screening out residues having significant differences, and extracting Cα atoms and atoms capable of forming a hydrogen bond, salt bridge or π-π stacking from the residues to construct a reference atom set;
screening out atoms capable of forming a hydrogen bond, salt bridge or π-π stacking interaction with atoms of the reference atom set from the M transition structures to construct a supplementary atom set;
based on the M transition structures, obtaining a stability evaluation result by evaluating the stability of the interaction between the reference atom set and the supplementary atom set; and
based on the stability evaluation result, screening out atoms that do not meet preset stability criteria from the supplementary atom set, and integrating the reference atom set and the screened supplementary atom set to obtain a key atom set.

In an optional embodiment, both the screening of the atoms of the reference atom set and the screening of the atoms of the supplementary atom set follow the same determination criteria regarding the hydrogen bond, salt bridge or π-π stacking interaction.

In an optional embodiment, there is one positively charged hydrogen atom between two negatively charged atoms, one of the negatively charged atoms serves as a hydrogen bond acceptor, and the other negatively charged atom serves as a hydrogen bond donor; and
when a cutoff distance between the hydrogen bond acceptor and the hydrogen bond donor is not more than 3.5 Å, and an angle formed by a line between the hydrogen bond donor and the hydrogen atom and a line between the hydrogen bond acceptor and the hydrogen bond donor is not more than 30°, it is determined that a hydrogen bond interaction may be formed between the atoms.

In an optional embodiment, when there is one strongly positively charged atom and one strongly negatively charged atom, and a cutoff distance between the strongly positively charged atom and the strongly negatively charged atom is not more than 4.5 Å, it is determined that a salt bridge interaction may be formed between the atoms.

In an optional embodiment, when there are two adjacent parallel aromatic rings and a distance between centroids of the two aromatic rings is not more than 4 Å, it is determined that a π-π stacking interaction may be formed between the atoms, and the aromatic ring includes a five-membered ring or a six-membered ring.

In an optional embodiment, after obtaining the key atom set, the method further includes: constructing a path collective variable based on the key atom set, and obtaining a free energy surface and transition state/metastable state information of a transition path, thereby providing a transition path microscopic mechanism of functional dynamics of biomolecules.

In an optional embodiment, the stability criteria include:
atoms that stably maintain a conformation number of not less than 2 for the hydrogen bond and salt bridge interactions; and
atoms where a distance between centroids of two aromatic rings does not change by more than 1 Å or an angle between the aromatic rings does not change by more than 30° for the π-π stacking interaction.

In an optional embodiment, the path collective variable is calculated as follows:

$$s = \frac{{\sum }_{i = 1}^{M} {ie}^{- λ {d}_{x , i}^{2}}}{{\sum }_{i = 1}^{M} {e}^{- λ {d}_{x , i}^{2}}}$$

$$z = - \frac{1}{λ} ln ( {\sum }_{i = 0}^{M} {e}^{- λ {d}_{x , i}^{2}} )$$

$$λ = 2.3 ( M - 1 ) / {\sum }_{i = 1}^{M - 1} {d}_{i , i + 1}^{2}$$

wherein M represents a number of transition structures including the initial state and the target state of the transition path; i represents a transition structure number; d_{x,i} represents a structural characteristic difference between transition structure x and transition structure i (such as RMSD); s is a position of transition structure x along the transition path; z is a distance of transition structure x from the transition path; and λ is a scaling parameter required to calculate s and z.

A system for searching a key atom set in functional dynamics of biomolecules includes:
a structure obtaining unit, configured to perform optimization to obtain a corresponding transition path based on an initial state and a target state of functional dynamics of biomolecules, and record M transition structures generated in the optimization process and including the initial state and the target state; wherein M is a natural number greater than 10;
a reference set unit, configured to, by comparing a structural difference between the initial state and the target state, screen out residues having significant differences, and extract Cα atoms and atoms capable of forming a hydrogen bond, salt bridge or π-π stacking from the residues to construct a reference atom set;
a supplementary set unit, configured to screen out atoms capable of forming a hydrogen bond, salt bridge or π-π stacking interaction with atoms of the reference atom set from the M transition structures to construct a supplementary atom set;
a stability evaluation unit, configured to, based on the M transition structures, obtain a stability evaluation result by evaluating the stability of the interaction between the reference atom set and the supplementary atom set; and
a key set unit, configured to, based on the stability evaluation result, screen out atoms that do not meet preset stability criteria from the supplementary atom set, and integrate the reference atom set and the screened supplementary atom set to obtain a key atom set.

In an optional embodiment, the system further includes:
a microscopic mechanism unit, configured to, construct a path collective variable based on the key atom set, and obtain a free energy surface and transition state/metastable state information of a transition path, thereby providing a transition path microscopic mechanism of functional dynamics of biomolecules.

Beneficial effects: the present disclosure provides a method and system for searching a key atom set in functional dynamics of biomolecules, which, based on a multi-screening method, quickly and accurately obtain a dominant key atom set in a transition path of functional dynamics of biomolecules, thereby greatly reducing the operations that rely on experience. The present disclosure may achieve parallel calculation of hydrogen bond/salt bridge/π-π stacking interaction, greatly reduce time cost, and is suitable for the transition path with excessive residues and interactions. The calculation method for the stability of hydrogen bond, salt bridge and π-π stacking interactions may reduce the use complexity of the method, lower the difficulty of obtaining key atom sets, and be extended to more complex biological systems.

### BRIEF DESCRIPTION OF DRAWINGS

FIG. 1 is a schematic diagram of an existing key atom set search process based on a transition path;

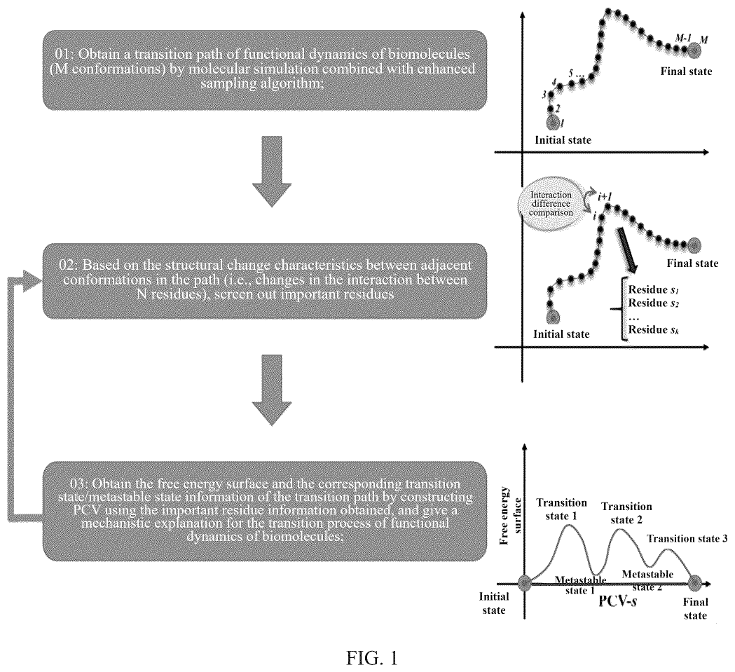

FIG. 2 is a schematic diagram of a flow chart of a method according to the present disclosure;

FIG. 3 is a schematic diagram of the calculation process of a transition path microscopic mechanism according to the present disclosure;

FIG. 4 is a schematic diagram of the principle of a method according to the present disclosure;

FIG. 5 is a schematic diagram of a calculation method for evaluating whether a hydrogen bond, salt bridge or π-π stacking interaction between residues is formed proposed by the present disclosure;

FIG. 6 is a schematic diagram of screening heavy atoms that form a hydrogen bond, salt bridge or π-π stacking interaction between residues from common biomolecule residues according to the present disclosure;

FIG. 7 is a schematic diagram of a calculation method for evaluating the stability of a hydrogen bond, salt bridge or π-π stacking interaction between residues according to the present disclosure; and

FIG. 8 is a schematic diagram of modules of the system according to the present disclosure.

To more clearly illustrate the technical solutions of embodiments of the present disclosure, the drawings required in the embodiments will be briefly described below. It should be understood that the following drawings only illustrate some embodiments of the present disclosure and therefore should not be considered as limitations of the scope, and for those of ordinary skill in the art, other related drawings can be obtained according to these drawings without creative efforts.

Reference numerals: 1. structure obtaining unit; 2. reference set unit; 3. supplementary set unit; 4. stability evaluation unit; 5. key set unit; and 6. microscopic mechanism unit.

### DETAILED DESCRIPTION OF EMBODIMENTS

Hereinafter, various embodiments disclosed in the present disclosure are described more fully. The present disclosure is disclosed as having various embodiments, and adjustments and modifications may be made therein. However, it should be understood that there is no intention to limit the various embodiments disclosed in the present disclosure to the specific embodiments disclosed herein, but rather the disclosure of the present disclosure should be construed to cover all modifications, equivalents and/or alternatives falling within the spirit and scope of the various embodiments disclosed in the present disclosure.

It should be noted that the multiple screening method is used in the present disclosure, and a reference atom set and a supplementary atom set are set for the convenience of description. In practical applications, instead of setting the reference atom set (RAS) and the supplementary atom set (SAS), all important residues of a plurality of domains may be directly predetermined, the stability of all hydrogen bond, salt bridge and π-π stacking interactions within these residues may be screened, and the transition path of the functional dynamics of biomolecules may be explained based on the obtained inaccurate key atom set, so as to give a transition mechanism.

The terms used in the various embodiments disclosed in the present disclosure are only for the purpose of describing the specific embodiments and are not intended to limit the various embodiments disclosed in the present disclosure. As used herein, the singular forms are intended to include the plural forms as well, unless the context clearly indicates otherwise. Unless otherwise defined, all terms (including technical and scientific terms) used herein have the same meaning as commonly understood by those ordinary skill in the art to which the various embodiments disclosed in the present disclosure belong. The terms (such as those defined in commonly used dictionaries) will be interpreted as having the same meaning as the contextual meaning in the relevant technical field and will not be interpreted as having an idealized meaning or an overly formal meaning unless clearly defined in various embodiments disclosed in the present disclosure.

### Embodiment 1

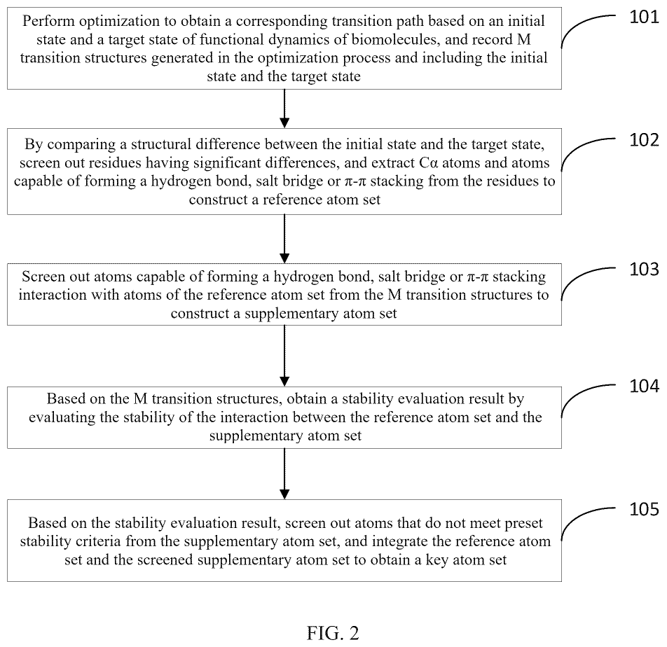

Embodiment 1 of the present disclosure discloses a method for searching a key atom set in functional dynamics of biomolecules, which quickly and accurately obtains a dominant key atom set in a transition path of functional dynamics of biomolecules, greatly reduces the operations that rely on experience, and greatly reduces the time costs. A flow chart of a method is shown in FIG. 2 of the specification, and the specific solution is as follows:

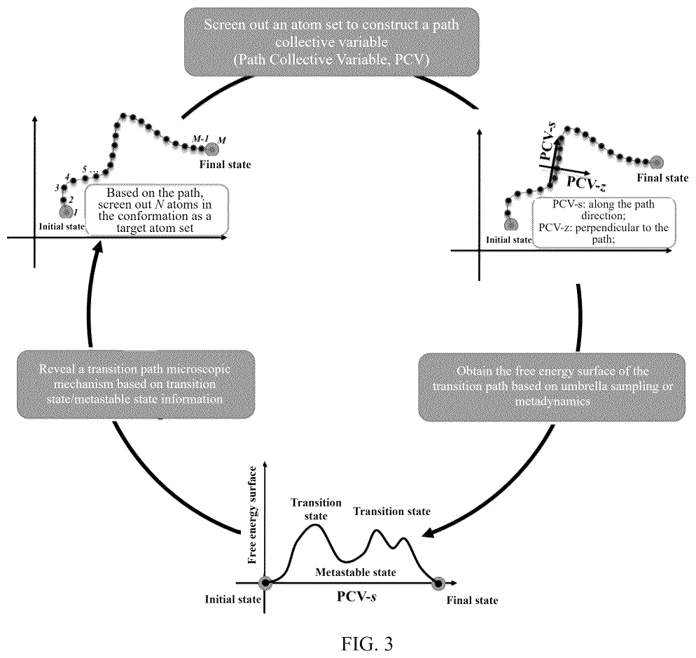

A method for searching a key atom set in functional dynamics of biomolecules includes:
101: performing optimization to obtain a corresponding transition path based on an initial state and a target state of functional dynamics of biomolecules, and recording M transition structures generated in the optimization process and including the initial state and the target state; wherein M is a natural number greater than 10;
102: by comparing a structural difference between the initial state and the target state, screening out residues having significant differences, and extracting Cα atoms and atoms capable of forming a hydrogen bond, salt bridge or π-π stacking from the residues to construct a reference atom set;
103: screening out atoms capable of forming a hydrogen bond, salt bridge or π-π stacking interaction with atoms of the reference atom set from the M transition structures to construct a supplementary atom set;
104: based on the M transition structures, obtaining a stability evaluation result by evaluating the stability of the interaction between the reference atom set and the supplementary atom set; and
105: based on the stability evaluation result, screening out atoms that do not meet preset stability criteria from the supplementary atom set, and integrating the reference atom set and the screened supplementary atom set to obtain a key atom set.
after obtaining the key atom set, the method further includes: constructing a path collective variable based on the key atom set, and obtaining a free energy surface and transition state/metastable state information of a transition path, thereby providing a transition path microscopic mechanism of functional dynamics of biomolecules. The specific process is shown in FIG. 3. First, based on the key atom set, a path collective variable (PCV) is constructed; then, the free energy surface calculation based on PCV is completed and the transition state/metastable state information is obtained; finally, a reasonable microscopic mechanism explanation is given for the transition path.

The process of screening out an atom set to construct PCV is as follows:

$$s = \frac{{\sum }_{i = 1}^{M} {ie}^{- λ {d}_{x , i}^{2}}}{{\sum }_{i = 1}^{M} {e}^{- λ {d}_{x , i}^{2}}}$$

$$z = - \frac{1}{λ} ln ( {\sum }_{i = 0}^{M} {e}^{- λ {d}_{x , i}^{2}} )$$

$$λ = 2.3 ( M - 1 ) / {\sum }_{i = 1}^{M - 1} {d}_{i , i + 1}^{2}$$

wherein M represents a number of transition structures including the initial state and the target state of the transition path; i represents a transition structure number; d_{x,i} represents a structural characteristic difference between transition structure x and transition structure i (such as RMSD); s is a position of transition structure x along the transition path; z is a distance of transition structure x from the transition path; and λ is a scaling parameter required to calculate s and z.

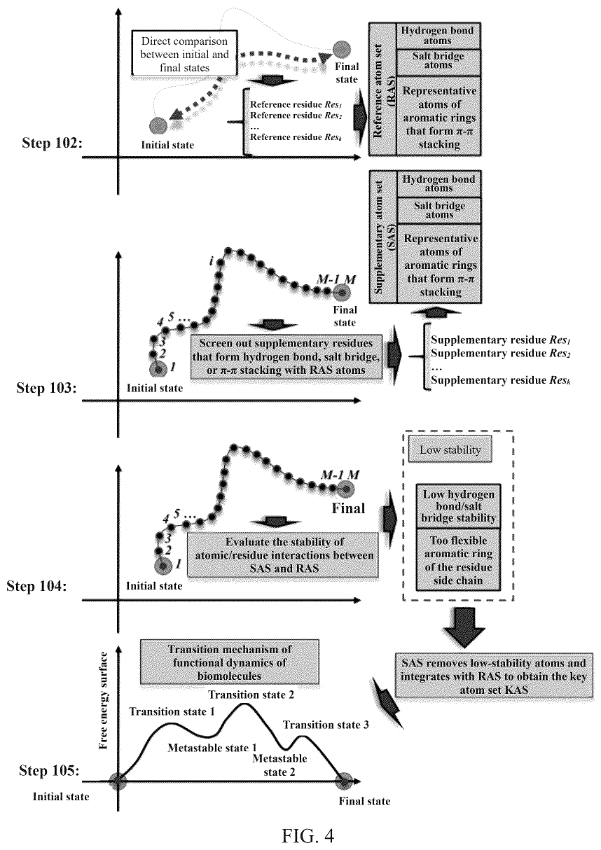

In this embodiment, a multi-screening method is applied, so that the difficulty in obtaining a dominant key atom set in a transition path of functional dynamics of biomolecules is reduced. Specifically, a reference atom set (RAS) is preset based on an initial state and a target state; based on the recognized important interactions within biomolecules (a hydrogen bond, salt bridge and π-π stacking interaction), a supplementary atom set (SAS) is obtained through a rapid search; then, the SAS is screened by examining the stability of important interactions (a hydrogen bond, salt bridge and π-π stacking interaction); and finally, the dominant key atom set in the transition path of functional dynamics of biomolecules is accurately obtained. The principal diagram of step 102 to step 105 is shown in FIG. 4 of the specification.

First, it is necessary to determine two key structures of functional dynamics of biomolecules, namely an initial state and a target state. Based on the initial state and the target state, optimization is performed to obtain a corresponding transition path, which includes M transition structures (including the initial state and the target state). A transition path may be understood as a continuous transition process (such as a certain angle slowly changing from 30° to 160°); but the transition is not continuous when actually presented but a gradual transition form like 30°, 32°, 34°, . . . , 158°, and 160°. These structures that show the transition process are the so-called M transition structures. M varies depending on an object being studied. The M values of different systems vary greatly, but are generally above 20. For more complex processes, M may be as high as around 300.

For the transition path of functional dynamics of biomolecules under study, when the transition reaches the target state from the initial state, the main structure of the biomolecule is stable, and the structure of a local region has significant differences. In this case, a range of residues to be examined may be reduced to the local region. The reference atom set is generally defined as a region of change where the difference is very important and significant. The region with the significant difference may be directly determined by comparing the initial state and the target state. However, in the specific transition process, there are often some other atoms that play an important role and cannot be ignored. This part of atoms can only be obtained based on the analysis of the transition path (M transition structures). In this embodiment, this part of atoms is integrated into a supplementary reference set. The reference atom set is obtained by extracting the initial state and the target state, which represents the part with significant differences; and the supplementary atom set is extracted based on M transition structures, which represents the atoms that play an important role in the transition process.

Both the screening of the atoms of the reference atom set and the screening of the atoms of the supplementary atom set follow the same determination criteria regarding the hydrogen bond, salt bridge or π-π stacking interaction. This embodiment designs a calculation method for the stability of the hydrogen bond, salt bridge and π-π stacking interaction in the transition path, and the design architecture ensures that parallel calculation (hydrogen bond/salt bridge/π-π stacking interaction may be calculated separately and simultaneously) is achieved, which may reduce use complexity and facilitate practical application in more complex biological systems.

For specific biomolecular system, such as membrane proteins, lipid molecules, small molecules and other special molecules, a specific hydrogen bond, salt bridge and π-π stacking interaction stability evaluation method is designed to improve accuracy and efficiency.

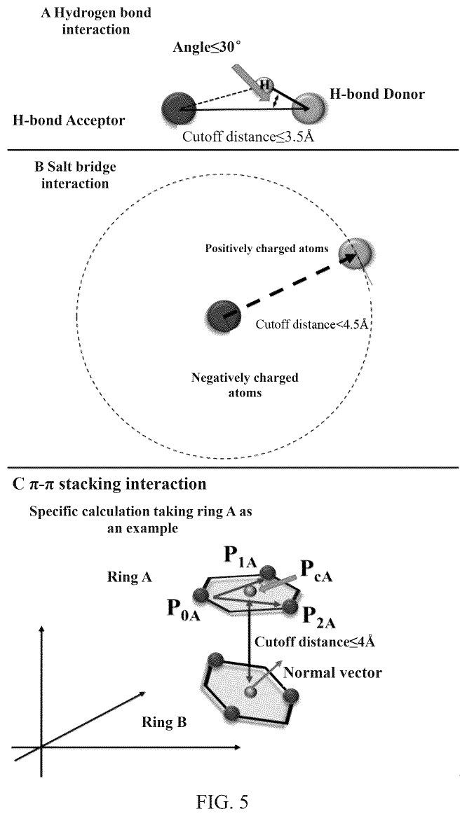

For the hydrogen bond interaction, the structure is specifically shown in FIG. 5 of the specification. There is one positively charged hydrogen atom between two negatively charged atoms, one of the negatively charged atoms serves as a hydrogen bond acceptor, and the other negatively charged atom serves as a hydrogen bond donor; and when a cutoff distance between the hydrogen bond acceptor and the hydrogen bond donor is not more than 3.5 Å, and an angle formed by a line between the hydrogen bond donor and the hydrogen atom and a line between the hydrogen bond acceptor and the hydrogen bond donor is not more than 30°, it is determined that the hydrogen bond interaction may be formed between the atoms.

For the salt bridge interaction, the structure is specifically shown in FIG. 5 of the specification. When there is one strongly positively charged atom and one strongly negatively charged atom, and a cutoff distance between the strongly positively charged atom and the strongly negatively charged atom is not more than 4.5 Å, it is determined that a salt bridge interaction may be formed between the atoms.

For the π-π stacking interaction, the structure is specifically shown in FIG. 5 of the specification. When there are two adjacent parallel aromatic rings and a distance between centroids of the two aromatic rings is not more than 4 Å, it is determined that a π-π stacking interaction may be formed between the atoms. The aromatic ring includes a five-membered ring or a six-membered ring. The π-π stacking interaction is mainly between the five-membered ring and the six-membered ring.

The distance between ring centroids is calculated as follows:

$${P}_{CA} = \frac{1}{3} ( {P}_{0 A} + {P}_{1 A} + {P}_{2 A} )$$

$${P}_{CB} = \frac{1}{3} ( {P}_{0 B} + {P}_{1 B} + {P}_{2 B} )$$

$${Distance}_{AB} = \vert {P}_{CA} - {P}_{CB} \vert$$

P_{0A}, P_{1A}, P_{2A} represent three atoms of the aromatic ring A, P_{0B}, P_{1B},

$${P}_{2 B}$$

represent three atoms of the aromatic ring B, and the distribution structure of the three atoms is shown in FIG. 5. P_{CA} and [symbol] are geometric centers of the aromatic ring A and the aromatic ring B, respectively, and [symbol] represents a distance between the geometric centers of the aromatic ring A and the aromatic ring B.

The angle between the rings is calculated as follows:

$${P}_{01 A} = {P}_{0 A} - {P}_{1 A}$$

$${P}_{02 A} = {P}_{0 A} - {P}_{2 A}$$

$$Norm . {Vec}_{A} = {P}_{01 A} \times {P}_{02 A}$$

$${P}_{01 B} = {P}_{0 B} - {P}_{1 B}$$

$${P}_{02 B} = {P}_{0 B} - {P}_{2 B}$$

$$Norm . {Vec}_{B} = {P}_{01 B} \times {P}_{02 B}$$

$${Angle}_{AB} = {cos}^{- 1} ( \frac{Norm . {Vec}_{A} \cdot Norm . {Vec}_{B}}{\vert Norm . {Vec}_{A} \vert \vert Norm . {Vec}_{B} \vert} )$$

wherein P_{01A} represents a coordinate vector difference between atom P_{0A} and atom P_{1A}, P_{01B} represents a coordinate vector difference between atom P_{0B} and atom P_{1B}, P_{02A} represents a coordinate vector difference between atom P_{0A} and atom P_{2A}, P_{02B} represents a coordinate vector difference between atom P_{0B} and atom, P_{2B}, Norm.Vec_{A} is a normal vector of the ring A, Norm. [symbol] is a normal vector of the ring B, and Angle_{AB} is an angle calculated based on the normal vectors of the ring A and the ring B.

Taking RAS as a reference, the residues that interact with RAS atoms are screened based on the determination conditions of the hydrogen bond, salt bridge and π-π stacking interaction, and the corresponding heavy atoms (non-hydrogen atoms) of these residues are extracted based on the criteria to construct a supplementary atom set (SAS). Under the full-atom force field, depending on the charge of the atoms, the atoms with particularly strong positive or negative charges are retained, and other atoms are ignored. In this embodiment, heavy atoms that may form the hydrogen bond, salt bridge or π-π stacking interaction between residues are extracted from common biomolecule residues. The heavy atoms in all residues in biological systems, which may be used to complete interactions such as hydrogen bonds, have been preset, and only the cutoff distance between atoms needs to be simply calculated.

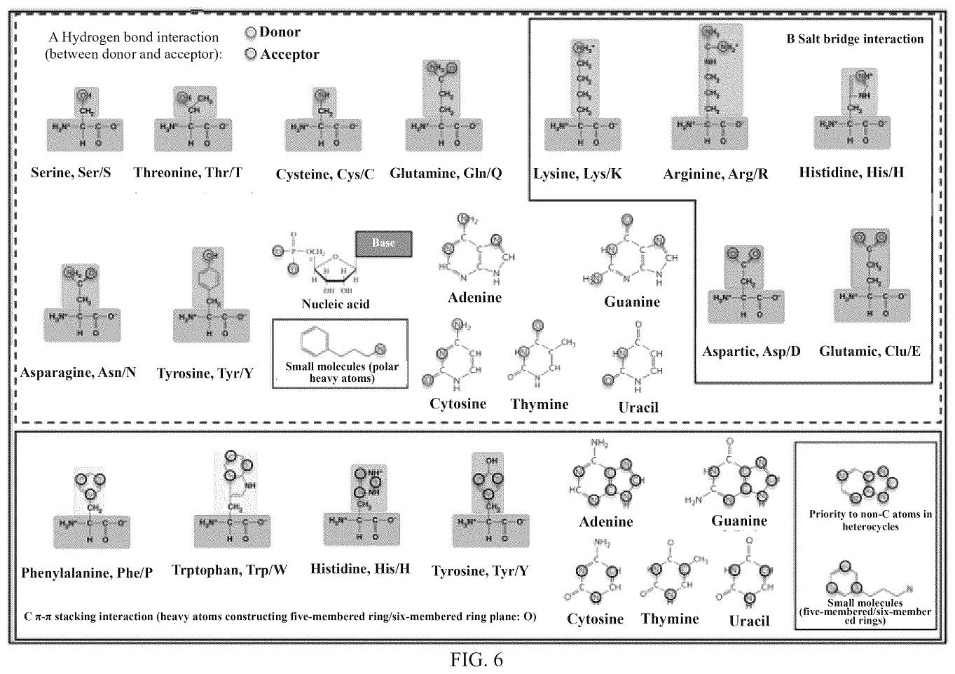

In the specification, FIG. 6 shows some examples of the hydrogen bond, salt bridge and π-π stacking interaction. Atoms that may form the hydrogen bond interaction include serine, threonine, cysteine, glutamic acid, aspartic acid, tyrosine, adenine, guanine, cytosine, thymine, and the like, and hydrogen bond donors and hydrogen bond acceptors are shown in FIG. 6. Atoms that may form the salt bridge interaction include lysine, arginine, histidine, aspartic acid, and glutamic acid. Atoms that may form π-π stacking interaction include phenylalanine, tryptophan, histidine, tyrosine, adenine, guanine, cytosine, thymine, and uracil.

Based on the M transition structures; a stability evaluation result is obtained by evaluating the stability of the interaction between the reference atom set and the supplementary atom set. The stability criteria include: atoms that stably maintain a conformation number of not less than 2 for the hydrogen bond and salt bridge interactions; and atoms where a distance between centroids of two aromatic rings does not change by more than 1 Å or an angle between the aromatic rings does not change by more than 30° for the π-π stacking interaction.

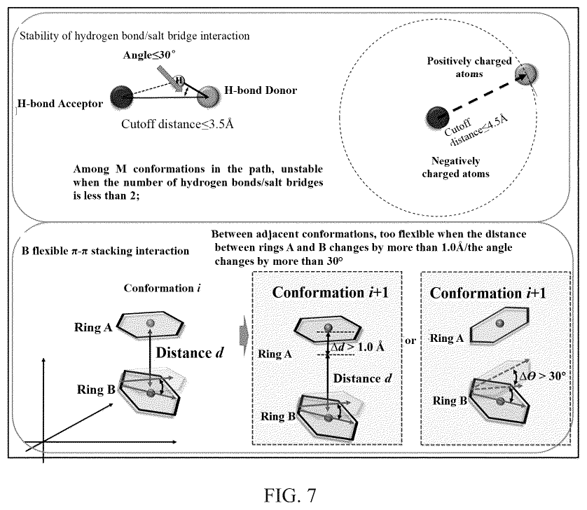

According to the stability evaluation result, structures with poor stability are removed. The stability determination criteria are shown in FIG. 7 of the specification. For hydrogen bond and salt bridge interaction, interactions with less than 2 stable conformations are screened and removed (corresponding atoms in SAS). For π-π stacking interaction, the changes in the angles and distances of the five-membered or six-membered ring planes of the corresponding residues along the adjacent structures in the transition path are calculated, and residues with angle changes greater than 30° or distance changes between the ring centroids greater than 1.0 Å are screened and removed. For angle calculation, the normal vector of the five-membered ring/six-membered ring is obtained, and then the vector is converted into the angle between the aromatic rings. It should be noted that the distance change between the ring centroids is greater than 1.0 Å, which is a change quantity rather than a simple distance value, and is referred to as Δa in FIG. 7.

Finally, the screened SAS is integrated with RAS to obtain the final dominant key atom set (KAS) to construct PCV, and a free energy surface and transition state/metastable state information of a transition path are obtained, thereby providing a transition path microscopic mechanism of functional dynamics of biomolecules.

After experimental verification, with the method of this embodiment, the key atom set screening dominated by three representative paths from the ground state to the activated state of 164-residue T4 lysozyme (T4 Lysozyme L99A, T4L L99A) has been completed, and a reasonable transition mechanism has been obtained. Furthermore, with the method of this embodiment, the key atom sets for the six representative transition pathways of the recognition and loading of target DNA single strands (21 bases) by the complex of the 685-residue thermophilic archaeal Argonaute protein and guide DNA single strand (21 bases) are successfully obtained. Based on this atom set, a relatively reasonable transition mechanism is also obtained.

This embodiment provides a method for searching a key atom set in functional dynamics of biomolecules, which, based on a multi-screening method, quickly and accurately obtain a dominant key atom set in a transition path of functional dynamics of biomolecules, thereby greatly reducing the operations that rely on experience. The present disclosure may achieve parallel calculation of hydrogen bond/salt bridge/π-π stacking interaction, greatly reduce time cost, and is suitable for the transition path with excessive residues and interactions. The calculation method for the stability of hydrogen bond, salt bridge and π-π stacking interactions may reduce the use complexity of the method, lower the difficulty of obtaining key atom sets, and be extended to more complex biological systems.

### Embodiment 2

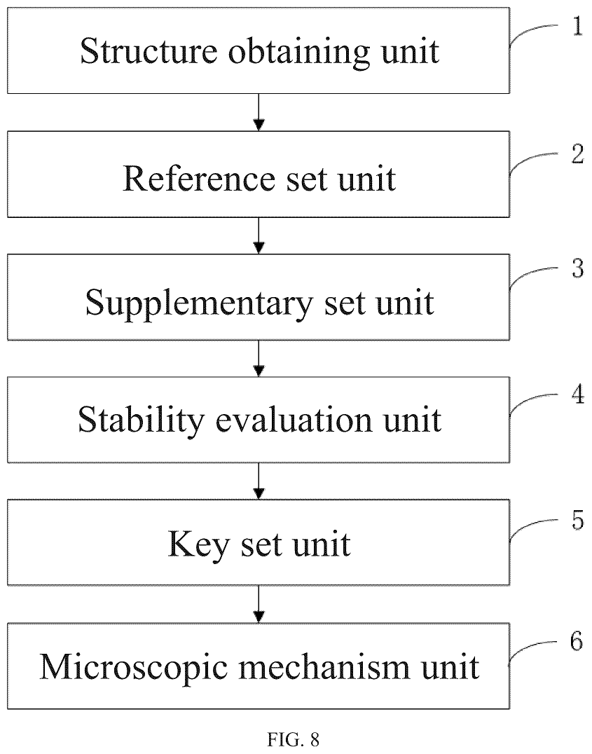

Embodiment 2 of the present disclosure discloses a system for searching a key atom set in functional dynamics of biomolecules. Based on Embodiment 1, the method of Embodiment 1 is systematized, the specific structure is shown in FIG. 8 of the specification, and the specific solution is as follows:

A system for searching a key atom set in functional dynamics of biomolecules includes:
a structure obtaining unit 1, configured to perform optimization to obtain a corresponding transition path based on an initial state and a target state of functional dynamics of biomolecules, and record M transition structures generated in the optimization process and including the initial state and the target state; wherein M is a natural number greater than 10;
a reference set unit 2, configured to, by comparing a structural difference between the initial state and the target state, screen out residues having significant differences, and extract Cα atoms and atoms capable of forming a hydrogen bond, salt bridge or π-π stacking from the residues to construct a reference atom set;
a supplementary set unit 3, configured to screen out atoms capable of forming a hydrogen bond, salt bridge or π-π stacking interaction with atoms of the reference atom set from the M transition structures to construct a supplementary atom set;
a stability evaluation unit 4, configured to, based on the M transition structures, obtain a stability evaluation result by evaluating the stability of the interaction between the reference atom set and the supplementary atom set;
a key set unit 5, configured to, based on the stability evaluation result, screen out atoms that do not meet preset stability criteria from the supplementary atom set, and integrate the reference atom set and the screened supplementary atom set to obtain a key atom set; and
a microscopic mechanism unit 6, configured to, construct a path collective variable based on the key atom set, and obtain a free energy surface and transition state/metastable state information of a transition path, thereby providing a transition path microscopic mechanism of functional dynamics of biomolecules.

In the reference set unit 2 and the supplementary set unit 3, atoms are screened based on the following principles.

For the hydrogen bond interaction, the structure is specifically shown in FIG. 5 of the specification. There is one positively charged hydrogen atom between two negatively charged atoms, one of the negatively charged atoms serves as a hydrogen bond acceptor, and the other negatively charged atom serves as a hydrogen bond donor; and when a cutoff distance between the hydrogen bond acceptor and the hydrogen bond donor is not more than 3.5 Å, and an angle formed by a line between the hydrogen bond donor and the hydrogen atom and a line between the hydrogen bond acceptor and the hydrogen bond donor is not more than 30°, it is determined that the hydrogen bond interaction may be formed between the atoms.

For the salt bridge interaction, the structure is specifically shown in FIG. 5 of the specification. When there is one strongly positively charged atom and one strongly negatively charged atom, and a cutoff distance between the strongly positively charged atom and the strongly negatively charged atom is not more than 4.5 Å, it is determined that a salt bridge interaction may be formed between the atoms.

For the π-π stacking interaction, the structure is specifically shown in FIG. 5 of the specification. When there are two adjacent parallel aromatic rings and a distance between centroids of the two aromatic rings is not more than 4 Å, it is determined that a π-π stacking interaction may be formed between the atoms, and the aromatic ring includes a five-membered ring or a six-membered ring. The π-π stacking interaction is mainly between the five-membered ring and the six-membered ring.

This embodiment provides a system for searching a key atom set in functional dynamics of biomolecules, which systematizes the method of Embodiment 1 to make this method more practical.

The present disclosure provides a method and system for searching a key atom set in functional dynamics of biomolecules, which, based on a multi-screening method, quickly and accurately obtain a dominant key atom set in a transition path of functional dynamics of biomolecules, thereby greatly reducing the operations that rely on experience. The present disclosure may achieve parallel calculation of hydrogen bond/salt bridge/π-π stacking interaction, greatly reduce time cost, and is suitable for the transition path with excessive residues and interactions. The calculation method for the stability of hydrogen bond, salt bridge and π-π stacking interactions may reduce the use complexity of the method, lower the difficulty of obtaining key atom sets, and be extended to more complex biological systems.

Those skilled in the art shall appreciate that the accompanying drawings are merely schematic diagrams of a preferred implementation scenario, and the modules or processes in the accompanying drawings are not necessarily required for implementing the present disclosure. Those skilled in the art can understand that the modules in the devices in the implementation scenario may be distributed in the devices in the implementation scenario according to the description of the implementation scenario, and may also be positioned in one or more devices different from this implementation scenario with corresponding changes. The modules in the above implementation scenarios can be combined into one module, or can be further divided into a plurality of sub-modules. The above serial numbers of the present disclosure are only for description and do not represent the advantages and disadvantages of the implementation scenarios. The above disclosure is only a few specific implementation scenarios of the present disclosure; however, the present disclosure is not limited thereto. Any changes that can be thought of by those skilled in the art should fall within the protection scope of the present disclosure.

What is claimed is:

1. A method for searching a key atom set in functional dynamics of biomolecules, comprising:
performing optimization to obtain a corresponding transition path based on an initial state and a target state of functional dynamics of biomolecules, and recording M transition structures generated in the optimization process and comprising the initial state and the target state; wherein M is a natural number greater than 10;
by comparing a structural difference between the initial state and the target state, screening out residues having significant differences, and extracting Cα atoms and atoms capable of forming a hydrogen bond, salt bridge or π-π stacking from the residues to construct a reference atom set;
screening out atoms capable of forming a hydrogen bond, salt bridge or π-π stacking interaction with atoms of the reference atom set from the M transition structures to construct a supplementary atom set;
based on the M transition structures, obtaining a stability evaluation result by evaluating the stability of the interaction between the reference atom set and the supplementary atom set; and
based on the stability evaluation result, screening out atoms that do not meet preset stability criteria from the supplementary atom set, and integrating the reference atom set and the screened supplementary atom set to obtain a key atom set.

2. The method according to claim 1, wherein both the screening of the atoms of the reference atom set and the screening of the atoms of the supplementary atom set follow the same determination criteria regarding the hydrogen bond, salt bridge or π-π stacking interaction.

3. The method according to claim 2, wherein there is one positively charged hydrogen atom between two negatively charged atoms, one of the negatively charged atoms serves as a hydrogen bond acceptor, and the other negatively charged atom serves as a hydrogen bond donor; and
when a cutoff distance between the hydrogen bond acceptor and the hydrogen bond donor is not more than 3.5 Å, and an angle formed by a line between the hydrogen bond donor and the hydrogen atom and a line between the hydrogen bond acceptor and the hydrogen bond donor is not more than 30°, it is determined that a hydrogen bond interaction is formed between the atoms.

4. The method according to claim 2, wherein when there is one strongly positively charged atom and one strongly negatively charged atom, and a cutoff distance between the strongly positively charged atom and the strongly negatively charged atom is not more than 4.5 Å, it is determined that a salt bridge interaction is formed between the atoms.

5. The method according to claim 2, wherein when there are two adjacent parallel aromatic rings and a distance between centroids of the two aromatic rings is not more than 4 Å, it is determined that a π-π stacking interaction is formed between the atoms, and the aromatic ring comprises a five-membered ring or a six-membered ring.

6. The method according to claim 1, wherein the stability criteria comprise:
atoms that stably maintain a conformation number of not less than 2 for the hydrogen bond and salt bridge interactions; and
atoms where a distance between centroids of two aromatic rings does not change by more than 1 Å or an angle between the aromatic rings does not change by more than 30° for the π-π stacking interaction.

7. The method according to claim 1, wherein after obtaining the key atom set, the method further comprises: constructing a path collective variable based on the key atom set, and obtaining a free energy surface and transition state/metastable state information of a transition path, thereby providing a transition path microscopic mechanism of functional dynamics of biomolecules.

8. The method according to claim 7, wherein the path collective variable is calculated as follows:
$$s = \frac{{\sum }_{i = 1}^{M} {ie}^{- λ {d}_{x , i}^{2}}}{{\sum }_{i = 1}^{M} {e}^{- λ {d}_{x , i}^{2}}}$$
$$z = - \frac{1}{λ} ln ( {\sum }_{i = 0}^{M} {e}^{- λ {d}_{x , i}^{2}} )$$
$$λ = 2.3 ( M - 1 ) / {\sum }_{i = 1}^{M - 1} {d}_{i , i + 1}^{2}$$
wherein M represents a number of transition structures comprising the initial state and the target state of the transition path; i represents a transition structure number; d_{x,i} represents a structural characteristic difference between transition structure x and transition structure i; s is a position of transition structure x along the transition path; z is a distance of transition structure x from the transition path; and λ is a scaling parameter required to calculate s and z.

9. A system for searching a key atom set in functional dynamics of biomolecules, comprising:
a structure obtaining unit, configured to perform optimization to obtain a corresponding transition path based on an initial state and a target state of functional dynamics of biomolecules, and record M transition structures generated in the optimization process and comprising the initial state and the target state; wherein M is a natural number greater than 10;
a reference set unit, configured to, by comparing a structural difference between the initial state and the target state, screen out residues having significant differences, and extract Cα atoms and atoms capable of forming a hydrogen bond, salt bridge or π-π stacking from the residues to construct a reference atom set;
a supplementary set unit, configured to screen out atoms capable of forming a hydrogen bond, salt bridge or π-π stacking interaction with atoms of the reference atom set from the M transition structures to construct a supplementary atom set;
a stability evaluation unit, configured to, based on the M transition structures, obtain a stability evaluation result by evaluating the stability of the interaction between the reference atom set and the supplementary atom set; and
a key set unit, configured to, based on the stability evaluation result, screen out atoms that do not meet preset stability criteria from the supplementary atom set, and integrate the reference atom set and the screened supplementary atom set to obtain a key atom set.

10. The system according to claim 9, further comprising:
a microscopic mechanism unit, configured to, construct a path collective variable based on the key atom set, and obtain a free energy surface and transition state/metastable state information of a transition path, thereby providing a transition path microscopic mechanism of functional dynamics of biomolecules.
